(19) United States
(12) Plant Patent Application Publication
(10) Pub. No.: US 2017/0311499 P1
(43) Pub. Date: Oct. 26, 2017

(54) Rose plant named 'RNF Pink 03WL'

(50) ;

(71) Applicant: Kaneya Co., Ltd., Chita (JP)

(72) Inventor: Hitoshi Nakashima, Wanochi-cho Ampachi-gun (JP)

(21) Appl. No.: 14/999,315

(22) Filed: Apr. 22, 2016

(51) Int. Cl. A01H 5/02

(52) U.S. Cl. CPC A01H 5/0222
USPC PLT/107

(57) ABSTRACT

A new and distinct rose plant having a strong fragrance and brink pink colored flowers with numerous petals.

Botanical classification: Rosa L.

Varietal denomination: ‘RNF Pink 03WL’

### BACKGROUND OF THE INVENTION

The present invention comprises a new and distinct cultivar of rose plant known by the varietal name ‘RNF Pink 03WL’. The new variety was discovered in April of 2010 in Gifu, Japan. The new variety is the result of a planned breeding program between an unnamed and unpatented Rosa L. variety (female parent) from the breeder's own collection and an unnamed and unpatented Rosa Rugosa variety (male parent) from the breeder's own collection. ‘RNF Pink 03WL’ has a different flower color than its female parent. Additionally, ‘RNF Pink 03WL’ exhibits a fragrance like its male parent, but differs from its male parent in its actual fragrance. When ‘RNF Pink 03WL’ is compared to Rosa L. ‘Ever172’ (currently unpatented in the U.S., Japanese Patent No. 20890), the varieties are similar in flower color, not having have eyes on their flowers, and in that they exhibit double flowers. However, when compared to ‘Ever172’, ‘RNF Pink 03WL’ has a larger flower size, different number of petals, and exhibits a fragrance. When generally compared to other rose varieties known to the breeder, the new variety differs in its strong fragrance, its type of fragrance, and in flower color. ‘RNF Pink 03WL’ has been trial and field tested and has been found to retain its distinctive characteristics and remain true to type through successive propagations.

### DESCRIPTION OF THE DRAWINGS

The accompanying photographic drawings illustrate the new variety, with the color being as nearly true as is possible with color illustrations of this type:

FIG. 1 shows a close-up view of a flower of the new variety;

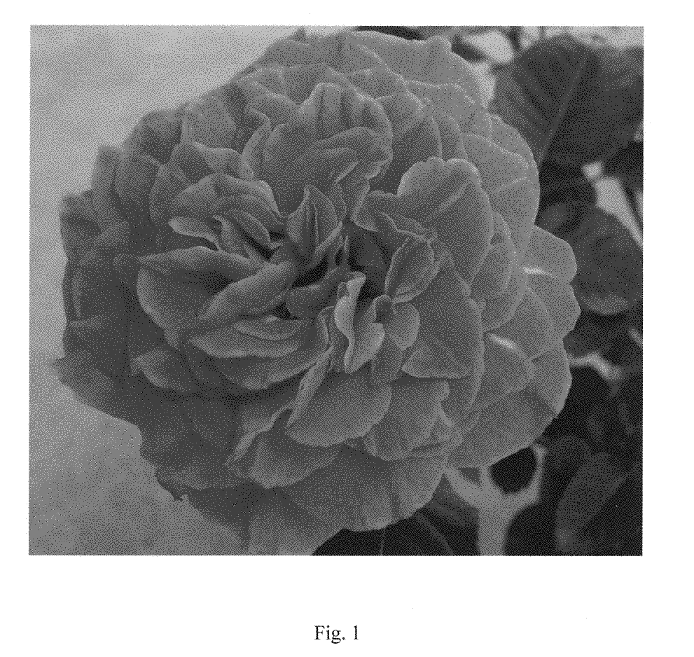

FIG. 2 shows a view of an entire plant of the new variety;

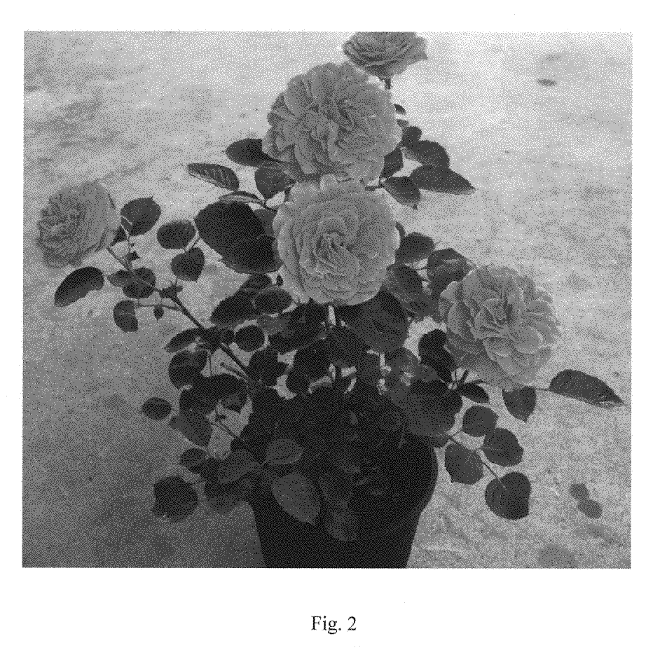

FIG. 3 shows a view of multiple plants of the new variety; and

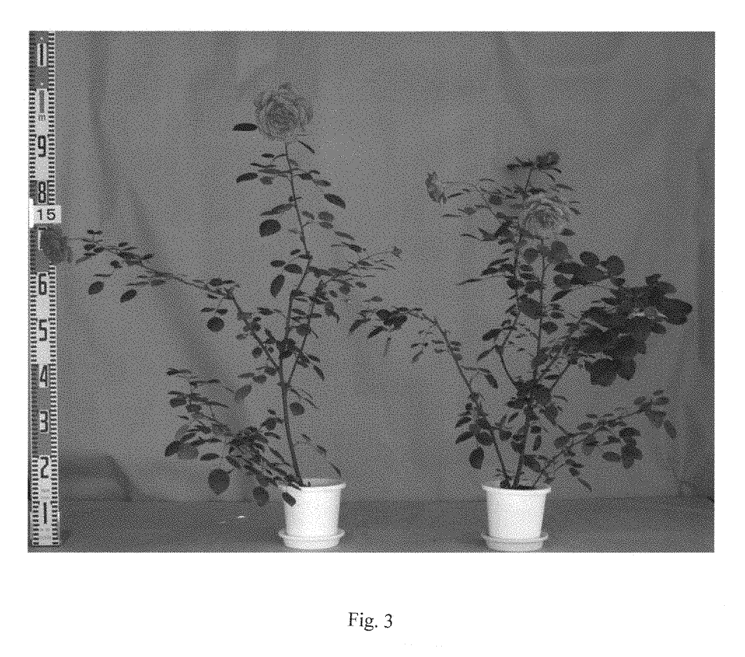

FIG. 4 shows the leaves of the new variety.

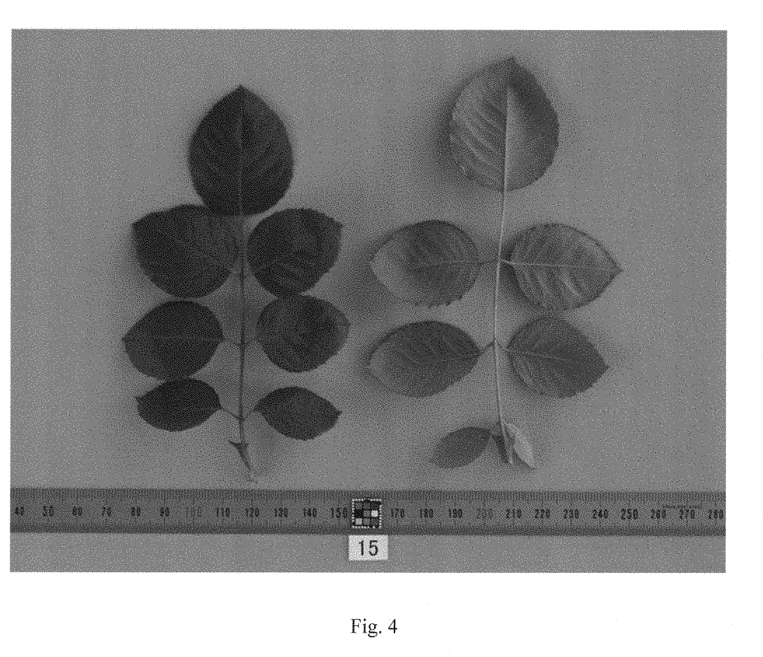

### DESCRIPTION OF THE PLANT

The following detailed description sets forth the characteristics of the new cultivar. The data which defines these characteristics were collected by asexual reproductions first performed via cuttings in May of 2011 carried out in Gifu, Japan. Plants of the new variety were grown in a greenhouse under natural light in 15 cm pots with peat moss substrate and with the assistance of heaters in the winter. The color readings and measurements were taken indoors in a greenhouse under natural light on one year old plants grown in containers. Color references are primarily to the R.H.S. Colour Chart of The Royal Horticultural Society of London.
Plant:
   Time to initiate roots.—About 21-28 days at approximately 25° C. Time to develop roots: About 30-45 days at approximately 25° C. Time to produce a finished flowering plant from a rooted cutting: About 5 months/20-22 weeks in a 15 cm container. Rooting habit: Similar to standard roses. Form: Upright. Distance from media surface to top of foliage: 30-35 cm at the end of production. Distance from media surface to top of flowers: 35-40 cm. Plant diameter: Around 25 cm. Vigor: Average. Strength (need for artificial support): No artificial support needed. Branching characteristics: Branching from bottom or lower level Stem Color: Immature: N57A. Mature: 132D. Surface texture: Young wood: Similar to standard roses. Older wood: Similar to standard roses. Average stem diameter: From 7 mm to 1 cm. Internode length: Approximately 4 to 5 cm. Number of internodes: 6 to 7. Thorns: Average number: Main branches: 50 per plant. Stems: 10 per stem. Length: 7 mm to 1 cm. Width: Around 5 mm. Color: Ivory/beige (8D) to red. Shape: Similar to standard roses. Foliage: Arrangement: Alternate. Average number of leaves per lateral branch: 7. Average number of leaflets per leaf: 7. Overall size of leaf: Length: 10 cm. Width: 5 cm. Leaflet: Shape of leaf (generally): Ovate and similar to standard roses. Shape of apex: Acute and similar to standard roses. Shape of base: Obtuse and similar to standard roses. Length range: 5-7 cm. Width range: 3-5 cm. Texture: Similar to standard roses. Margin type: Serrate and similar to standard roses. Color: Young leaves: Upper surface: 141C with a red edge. Lower surface: 145B with a red edge. Mature leaves: Upper surface: 125A. Lower surface: 130B. Veins: Venation type: Pinnate and similar to standard roses. Color: 138D. Petiole: Length: 2-3 mm. Diameter: 1-2 mm. Color: 139D with a hint of red. Texture: Similar to standard roses. Stipule: None present.
Inflorescence:
   Buds.—Number of buds per plant: 10. Number of buds per flowering stem: 2. Shape of individual bud: Napiform and similar to standard roses. Length of individual bud: 1.5-2 cm. Width of individual bud: 8 mm to 1 cm. Bud color: Green with red. Natural flowering season at specified location: End of April to May in Gifu, Japan. Average number of flowers per year at specified location: 10 to 15. Flowering habit: Continuous and freely flowering. Flower description: 2 or more flowers and bloom like a bunch. Lastingness of an individual flower on the plant: 10 days to 2 weeks. Lastingness of a cut flower: 1 week. Flower: General appearance: Double flowering rose. Shape: Round and mounded. Diameter: 8-10 cm. Height: 3-4 cm. Petals: Color: Upper surface (when opening): 67A. Lower surface (when opening): Pale bright pink. Upper surface (fully opened): 67A. Lower surface (fully opened): Pale bright pink with white. Shape: Rounded with a wavy habit. Length: 4-5 cm. Width: 5-6 cm. Apex shape: Round with a wide lip. Base shape: Round and narrow. Margin: Entire. Number per flower: 70. Texture: Soft and similar to standard rose petals. Reflex characteristics: None. Presence of basal petal spots: None. Petaloid description: Lightly fringed with semi-double flowering. Sepals: Color: Upper and lower surfaces (when opening): Green. Upper and lower surfaces (fully opened): Green. Shape: Sharp. Length: 2-3 cm. Width: 8 mm to 1 cm. Apex shape: Pointed. Base shape: Wide. Margin: Entire and tight, when closed. Number per flower: 4-5. Texture: Similar to standard roses. Pedicel: Length: 3-5 cm. Diameter: 3 mm. Texture: Similar to standard roses. Strength: Strong. Color: 142B. Peduncle color: 141D. Receptacle: Texture: Same as a standard Floribunda-type rose. Shape: Same as a standard Floribunda-type rose. Height: 5-7 mm. Width: 5-7 mm. Color: Green. Fragrance: Strong. Disease/pest resistance: Average, nothing unusual noted to date. Winter temperature tolerance: Blooming at 1° C. in winter. With only roots, −15˜-18° C. Hot temperature tolerance: 40° C. Drought tolerance: Average.
   Reproductive organs.—Stamens: Number (per flower): 50. Filament: Length: 7-10 mm. Color: Pale cream to white. Anthers: Shape: Like a match stick. Length: 2-3 mm. Color: Light yellow/beige. Pollen: Color: 10D. Amount (generally): Less than average. Pistils: Number: 30. Length: 1-2 mm. Style: Length: Average. Color: Creamy yellow. Stigma: Shape: Similar to standard roses. Color: Creamy yellow/beige. Size: 1 mm. Ovaries: Length: 3 mm. Width: 0.5 mm. Color: White.

I claim:

1. A new and distinct variety of rose plant, as is herein illustrated and described.
